(19) United States
(12) Patent Application Publication
(10) Pub. No.: US 2007/0197664 A1
(43) Pub. Date: Aug. 23, 2007

(54) Prevention and treatment of androgen-deprivation induced osteoporosis

(76) Inventors: Mitchell S. Steiner, Germantown, TN (US); Sharan Raghow, Collierville, TN (US); Karen A. Veverka, Cordova, TN (US)

Correspondence Address: PEARL COHEN ZEDEK LATZER, LLP, 1500 BROADWAY 12TH FLOOR, NEW YORK, NY, 10036, US

(21) Appl. No.: 11/656,566

(22) Filed: Jan. 23, 2007

Related U.S. Application Data
(63) Continuation-in-part of application No. 11/329,393, filed on Jan. 11, 2006.
Application No. 11/329,393 is a continuation-in-part of application No. 10/944,465, filed on Sep. 20, 2004.
Application No. 10/944,465 is a continuation-in-part of application No. 10/778,334, filed on Feb. 17, 2004.
Application No. 10/778,334 is a continuation-in-part of application No. 10/609,684, filed on Jul. 1, 2003, now abandoned.
Application No. 10/609,684 is a continuation-in-part of application No. 10/305,363, filed on Nov. 27, 2002, now Pat. No. 6,899,888.
(63) Continuation-in-part of application No. 10/778,333, filed on Feb. 17, 2004.
Application No. 10/778,333 is a continuation-in-part of application No. 10/747,691, filed on Dec. 30, 2003.
Application No. 10/747,691 is a continuation-in-part of application No. 10/609,684, filed on Jul. 1, 2003, now abandoned.
Application No. 10/609,684 is a continuation-in-part of application No. 10/305,363, filed on Nov. 27, 2002, now Pat. No. 6,899,888.
(60) Provisional application No. 60/333,734, filed on Nov. 29, 2001.
(60) Provisional application No. 60/333,734, filed on Nov. 29, 2001.

(51) Int. Cl. A61K 31/135

(52) U.S. Cl. 514/649

(57) ABSTRACT

This invention provides a method of treatment, prevention, suppression, inhibition, or reduction of risk of developing androgen-deprivation induced skeletal-related events (SRE), such as pathologic fractures, surgery to bone, radiation to bone, spinal cord compression, change in antineoplastic therapy, including changes in hormonal therapy, new bone metastases, bone loss, or a combination thereof in men suffering from prostate cancer, comprising administering to a male subject suffering from prostate cancer a selective estrogen receptor modulator (SERM) and/or its analog, derivative, isomer, metabolite, pharmaceutically acceptable salt, pharmaceutical product, hydrate, N-oxide, or any combination thereof.

### CROSS REFERENCE TO RELATED APPLICATION

This application is a continuation-in-part of U.S. application Ser. No. 11/329,393, filed Jan. 11, 2006, which is a continuation-in-part of U.S. application Ser. No. 10/944,465, filed Sep. 20, 2004, which is a continuation-in-part of U.S. application Ser. No. 10/778,334, filed Feb. 17,2004, which is a continuation-in-part of U.S. application Ser. No. 10/609,684, filed Jul. 3,2003, which is a continuation-in-part of U.S. application Ser. No. 10/305,363, filed Nov. 27, 2002, and claims priority of U.S. Provisional Application Ser. No. 60/333,734, filed Nov. 29, 2001. This application is also a continuation-in-part of U.S. application Ser. No. 10/778,333, filed Feb. 17,2004, which is a continuation-in-part of U.S. application Ser. No. 10/747,691, filed Dec. 30, 2003, which is a continuation-in-part application of U.S. application Ser. No. 10/609,684, filed Jul. 1, 2003, which is a continuation-in-part application of U.S. application Ser. No. 10/305,363, filed Nov. 27, 2002, and claims priority of U.S. Provisional Application Ser. No. 60/333,734, filed 29 Nov. 2001. These applications are incorporated by reference herein in their entirety.

### FIELD OF INVENTION

This invention relates to the treatment, prevention, suppression or inhibition of, or the reduction of the risk of developing a skeletal-related event (SRE), such as bone fractures, surgery of the bone, radiation of the bone, spinal cord compression, new bone metastasis, bone loss, or a combination thereof in a subject with cancer, comprising administering to the a selective estrogen receptor modulator (SERM) and/or its analog, derivative, isomer, metabolite, pharmaceutically acceptable salt, pharmaceutical product, hydrate, N-oxide, or any combination thereof. The invention relates, inter alia to treatment of an SRE with toremifene in a subject with prostate cancer undergoing or having undergone androgen deprivation therapy (ADT).

### BACKGROUND OF THE INVENTION

A number of pathologies result or manifest in damage to skeletal tissue. Collectively, such effects can be referred to as skeletal-related events (SRE). Skeletal-related events (SRE) include inter-alia pathologic fractures, spinal cord compression, hypercalcemia, and severe bone pain. One example of such a pathology resulting or manifesting in SRE is tumor metastasis to bone.

Metastatic bone disease may remain confined to the skeleton. In these circumstances, the decline in quality of life and eventual death is due almost entirely to skeletal complications and their subsequent treatment. Bone is typically the first, most frequent and often the only site of metastasis in patients with advanced prostate cancer, with a median survival following diagnosis of bone metastases in the range of 12 to 53 months. Bone is also a relatively frequent site for symptomatic metastases in patients with other solid tumors, including lung, thyroid, renal, and bladder cancers, of whom up to 40% of those presenting with bone metastases at cancer diagnosis, developing an ongoing risk of skeletal morbidity and often experiencing severe bone pain. Approximately 20% of patients with renal cell carcinoma are metastatic, with up to 35% of which, develop bone metastases during disease progression. Metastatic bone disease interferes with the coupled process of osteoclast-mediated bone resorption and osteoblast-mediated bone formation, which are involved in repair and maintenance of normal bone tissue. The result is often increased however imbalanced bone turnover that leads to a loss of structural integrity and consequent skeletal complications and skeletal-related events (SRE).

It is well established that the bone mineral density of males decreases with age. Decreased bone mineral content and density correlates with decreased bone strength and predisposes the bone to fracture. Sex-hormones appear to play a role in bone homeostasis, with physiologic concentrations of androgens and estrogens being involved in maintaining bone health, throughout adult life. Sex hormone deprivation typically results in an increase in the rate of bone remodeling, skewing the normal balance between bone resorption and formation to favor resorption, contributing to an overall loss of bone mass

Prostate cancer is one of the most frequently diagnosed noncutaneous cancers among men in the United States. One of the approaches to the treatment of prostate cancer is androgen deprivation in the subject. The male sex hormone, testosterone, stimulates the growth of cancerous prostatic cells and, therefore, is the primary fuel for the growth of prostate cancer. The goal of androgen deprivation is to decrease stimulation of cancerous prostatic cells by testosterone. Testosterone is normally produced by the testes in response to stimulation from a hormonal signal called luteinizing hormone (LH) which in turn is stimulated by luteinizing-hormone releasing hormone (LH-RH). Androgen deprivation in male subjects has been accomplished surgically, by bilateral orchidectomy, and chemically, for example, via the administration of LH-RH agonists (LHRHα) and/or antiandrogens.

Androgen deprivation in patients with micrometastatic disease has been shown to prolong survival [Messing EM, et al (1999), N Engl J Med 34, 1781-1788; Newling (2001), Urology 58(Suppl 2A), 50-55]. Moreover, androgen deprivation is being employed in numerous new clinical settings, including neoadjuvant therapy prior to radical prostatectomy, long-term adjuvant therapy for patients at high risk for recurrence following radiation or surgery, neoadjuvant therapy for radiation, and treatment of biochemical recurrence following radiation or surgery [Carroll, et al (2001), Urology 58, 1-4; Horwitz EM, et al (200 1), Int J Radiat Oncol Biol Phy Mar 15;49(4), 947-56]. Thus, more prostate cancer patients have become candidates for and are being treated by androgen ablation, and at an earlier time and for a prolonged period of time, than previously undertaken. Treatment lasting 10 or more years with androgen deprivation therapy is not uncommon.

Unfortunately, androgen deprivation therapy is accompanied by significant side effects, including hot flashes, gynecomastia, osteoporosis, decreased lean muscle mass, depression and other mood changes, loss of libido, and erectile dysfunction [Stege R (2000), Prostate Suppl 10,38-42]. Consequently, complications of androgen blockade now contribute significantly to the morbidity, and in some cases the mortality, of men suffering from prostate cancer.

Given that more patients today are being treated by long-term androgen deprivation, osteoporosis has become a clinically important side effect in men suffering from prostate cancer undergoing androgen deprivation. Loss of bone mineral density (BMD) occurs within 6 months in the majority of patients being treated by androgen deprivation. New innovative approaches are urgently needed to decrease the incidence of androgen-deprivation induced osteoporosis and bone disease in men suffering from prostate cancer.

### SUMMARY OF THE INVENTION

In one embodiment, this invention provides a method of preventing, treating, suppressing, inhibiting or reducing the risk of, reducing the incidence of, ameliorating symptoms, delaying progression, diminishing pathogenesis of a skeletal-related event (SRE) in a subject suffering from cancer, said method comprising the step of administering toremifene, or a pharmaceutically acceptable salt thereof to said subject.

In another embodiment, this invention provides a method of preventing, treating, suppressing, inhibiting or reducing the risk of, reducing the incidence of, ameliorating symptoms, delaying progression, diminishing pathogenesis of developing a skeletal-related event (SRE) in a male subject suffering from prostate cancer, said method comprising the step of administering a selective estrogen receptor modulator (SERM), or a pharmaceutically acceptable salt thereof to said subject.

In one embodiment, this invention provides a method of preventing, treating, suppressing, inhibiting or reducing the risk of, reducing the incidence of, ameliorating symptoms, delaying progression, diminishing pathogenesis of developing a skeletal-related event (SRE) in a male subject suffering from prostate cancer, said method comprising the step of administering toremifene, or a pharmaceutically acceptable salt thereof to said subject.

In another embodiment, this invention provides a method of preventing, treating, suppressing, inhibiting or reducing the risk of developing skeletal-related events (SRE) in a male subject suffering from prostate cancer, said method comprising the step of administering toremifene, its analogue, derivative, metabolite or a pharmaceutically acceptable salt thereof to said subject.

In one embodiment, the skeletal-related events are a product of cancer therapy. In one embodiment, the skeletal-related events are a product of androgen deprivation therapy. In one embodiment, the skeletal-related events are a pathologic fracture, a necessity for surgery of the bone, a necessity for radiation of the bone, spinal cord compression, new bone metastasis, bone loss, or a combination thereof.

In another embodiment, the subject demonstrates enhanced cancer pathogenesis proximal to, or prior to administering a selective estrogen receptor modulator (SERM). In one embodiment, the subject requires a change in antineoplastic therapy.

### DETAILED DESCRIPTION OF THE INVENTION

This invention provides, in some embodiments, methods of 1) treating SRE in a subject with cancer; 2) preventing SRE in a subject with cancer; 3) suppressing, inhibiting or reducing the risk of developing SRE in a subject with cancer by administering a selective estrogen receptor modulator (SERM) or a pharmaceutically acceptable salt thereof to the subject. In one embodiment, the SERM is toremifene, while in another embodiment, it is raloxifene or tamoxifen.

In one embodiment, provided herein is a method of preventing, treating, suppressing, inhibiting or reducing the risk of, reducing the incidence of, ameliorating symptoms, delaying progression, diminishing pathogenesis of a skeletal-related event (SRE) in a subject suffering from cancer, comprising the step of administering to the subject a composition comprising toremifene, raloxifene, tamoxifen or their analogue, functional derivative, metabolite or a combination thereof, or a pharmaceutically acceptable salt thereof to said subject. In one embodiment, the metabolite used in the compositions provided herein, or as utilized in the methods provided herein for the treatment of an SRE, comprise ospemifene, fispemifene or their combination.

In other embodiments, this invention provides methods of 1) treating SRE in a subject with cancer; 2) preventing SRE in a subject with cancer; 3) suppressing, inhibiting or reducing the risk of developing SRE in a subject with cancer by administering torem ifene or a pharmaceutically acceptable salt thereof to the subject.

In some embodiments, the subject is human, while in other embodiments, the subject is non-human. In some embodiments, the subject is mammalian. In one embodiment, the subject is simian, bovine, feline, canine, ovine, porcine, equine, or murine.

In one embodiment, the subject is male, while in another embodiment, the subject is female. In one embodiment, the subject suffers from prostate cancer.

In one embodiment, the skeletal-related events treated using the methods provided herein and/or utilizing the compositions provided herein, are fractures, which in one embodiment, are pathological fractures, non-traumatic fractures, vertebral fracture, non-vertebral fractures, morphometric fractures, or a combination thereof. In some embodiments, fractures may be simple, compound, transverse, greenstick, or comminuted fractures. In one embodiment, fractures may be to any bone in the body, which in one embodiment, is a fracture in any one or more bones of the arm, wrist, hand, finger, leg, ankle, foot, toe, hip, collar bone, or a combination thereof.

In another embodiment, the methods and/or compositions provided herein, are effective in treatment, prevention, suppression, inhibition or reduction of the risk of skeletal-related events such as pathologic fractures, spinal cord compression, hypercalcemia, bone-related pain, or their combination.

In another embodiment, the skeletal-related events sought to be treated using the methods provided herein and/or utilizing the compositions provided herein, comprise the necessity for bone surgery and/or bone radiation, which in some embodiments, is for the treatment of pain resulting in one embodiment from bone damage, or nerve compression. In another embodiment, the skeletal-related events sought to be treated using the methods provided herein and/or utilizing the compositions provided herein, comprise spinal cord compression, or the necessity for changes in antineoplastic therapy, including changes in hormonal therapy, in a subject. In some embodiments, skeletal-related events sought to be treated using the methods provided herein and/or utilizing the compositions provided herein, comprise treating, suppressing, preventing, reducing the incidence of, or delaying progression or severity of bone metastases, or bone loss. In one embodiment, bone loss may comprise osteoporosis, osteopenia, or a combination thereof. In one embodiment, skeletal-related events may comprise any combination of the embodiments listed herein.

In one embodiment, the methods provided herein and/or utilizing the compositions provided herein, are effective in reducing metastases to the bone, such as in terms of number of foci, the size of foci, or a combination thereof. According to this aspect of the invention and in one embodiment, provided herein is a method of preventing or inhibiting cancer metastasis to bone in a subject, comprising the step of administering to the subject a composition comprising toremifene, raloxifene, tamoxifen or an analogue, functional derivative, metabolite or a combination thereof, or a pharmaceutically acceptable salt thereof. In one embodiment, such metabolites may comprise ospemifene, fispemifene or their combination. In one embodiment, the cancer is is prostate cancer.

A person skilled in the art would readily recognize that changes in the antineoplastic therapy according to the methods provided herein, utilizing the compositions provided herein may be conducted as a function of, or adjusted or varied as a function of, inter-alia, the severity of the underlying disease, the source of the underlying disease, the extent of the patients' pain and source of the patients' pain, as well as the stage of the disease. The therapeutic changes may include in certain embodiments, changes in the route of administration (e.g. intracavitarily, intraartiarly, intratumoraly etc.), forms of the compositions administered (e.g. tablets, elixirs, suspensions etc.), changes in dosage and the like. Each of these changes are well recognized in the art and are encompassed by the embodiments provided herein.

In one embodiment, the skeletal-related events are a result of cancer therapy. In one embodiment, the skeletal-related events are a result of hormone deprivation therapy, while in another embodiment, they are a product of androgen deprivation therapy (ADT).

In males, while the natural decline in sex-hormones at maturity (direct decline in androgens as well as lower levels of estrogens derived from peripheral aromatization of androgens) is associated with the frailty of bones, this effect is more pronounced in males who have undergone androgen deprivation therapy.

As provided herein, the results demonstrate that administration of a SERM, such as, for example, toremifene, at a daily dosage of approximately 80 mg, decreases adverse skeletal related events, as is described in the Example hereinbelow.

“Estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-(2,2-dimethyl-1-oxopropoxy-4-methyl-2-[4-[2-(1-piperidinyl)ethoxy]ph-enyl]-2H-1-benzopyran-3-yl]-phenyl-2,2-dimethylpropanoate, 4,4′-dihydroxybenzophenone-2,4-dinitrophenyl-hydrazone, and SH646.

Toremifene is an example of a triphenylalkylene compound described in U.S. Pat. Nos. 4,696,949 and 5,491,173 to Toivola et al., the disclosures of which are incorporated herein by reference. Formulations containing toremifene are described, for example, in U.S. Pat. No.5,571,534 to Jalonen et al. and in U.S. Pat. No. 5,605,700 to DeGregorio et al., the disclosures of which are incorporated herein by reference.

Raloxifene (6-hydroxy-2-(4-hydroxyphenyl)-3-[4-(2-piperidinoethoxy)benzoyl]benzo[b]thiophene), is an example of a benzothiophene compound described in U.S. Pat. No 4,418,068 to Jones et al. Raloxifene competitively inhibits estrogen action in a number of in vitro and in vivo models (Black, Jones, and Falcone, Life Sci., 32,1031-1036 (1983); Knecht, Tsai-Morris, and Catt, Endocrinology, 116, 1771-1777 (1985); and Simard and Labrie, Mol. Cell. Endocrinology, 39, 141-144 (1985)). This compound also displays some estrogen-like actions in addition to its estrogen-antagonistic effects (Ortmann, Emons, Knuppen, and Catt, Endocrinology, 123, 962-968 (1988)). A recent report suggests that raloxifene is useful in the treatment of osteoporosis in postmenopausal women (Turner, Sato, and Bryant, Journal of Clinical Investigation (In Press)). Pharmaceutical formulations containing raloxifene are described in U.S. Pat. Nos. 5,972,383 and 5,811,120, and in European Patent No. 670162 to Gibson et al.

In one embodiment, the methods of this invention are directed to use of a SERM, such as, for example, toremifene treatment, prevention, suppression, inhibition or reduction of the risk of developing an SRE, which may comprise osteoporosis and/or loss of BMD and/or a bone fracture, which in some embodiments is a function of androgen-deprivation induced.

In another embodiment, the SERM for use in any method and/or composition of this invention comprises toremifene, raloxifene, tamoxifen or their analogue, functional derivative, metabolite or a combination thereof, or a pharmaceutically acceptable salt thereof. In one embodiment, the metabolite of toremifene, raloxifene, tamoxifen used in the compositions provided herein, as utilized in the methods provided herein for the prevention of bone metastases, is ospemifene, fispemifene or their combination. In one embodiment, ospemifene (FC-1271a; Z-2-[4-(4-chloro-1,2-diphenyl-but-1-enyl)phenoxy]ethanol), or an E isomer thereof, is used in the methods and/or compositions provided herein.

In one embodiment, fispemefene ((Z)-2-{2-[4-(4-Chloro-1,2-diphenylbut-1-enyl)phenoxy]ethoxy}ethanol), or its metabolite or pharmaceutically acceptable salt is used in the compositions and methods described herein, for the prevention or inhibition of bone metastases or treatment of SRE as provided herein.

In some embodiments, a dose of 80 mg/day of toremifene in humans is effective in decreasing adverse SREs. In one embodiment, the dosage is administered over a prolonged period of time. In one embodiment, the treatment is provided for 1 month, or in another embodiment, for 1-6 months, or in another embodiment, for 1-12 months, or in another embodiment, for at least one year, or in another embodiment, for the duration of androgen deprivation therapy. In another embodiment, the treatment is continuous, or in another embodiment, the treatment is cyclic, with specified periods of treatment and lack of treatment. In another embodiment, treatment is continued and discontinued as a function of bone density or bone mineral loss, such that the subject is evaluated at specified periods, and the administration regimen is tailored to individual responses to treatment.

In one embodiment, this invention provides a method of preventing, treating, suppressing, inhibiting or reducing the risk of developing skeletal-related events (SRE) in a subject suffering from cancer, said method comprising the step of administering a selective estrogen receptor modulator (SERM), or a pharmaceutically acceptable salt thereof to said subject.

In one embodiment, provided herein is a method of treating, reducing the incidence or severity of, or reducing the risk of developing a skeletal-related event (SRE) in a male subject suffering from cancer, said method comprising the step of administering toremifene, or a pharmaceutically acceptable salt thereof, or a metabolite thereof to said subject.

In another embodiment, this invention provides a method of preventing, treating, suppressing, inhibiting or reducing the risk of developing skeletal-related events (SRE) in a male subject suffering from prostate cancer, said method comprising the step of administering a selective estrogen receptor modulator (SERM), or a pharmaceutically acceptable salt thereof to said subject.

In another embodiment, this invention provides a method of preventing, treating, suppressing, inhibiting or reducing the risk of developing skeletal-related events (SRE) in a human male subject suffering from prostate cancer, said method comprising the step of administering a selective estrogen receptor modulator (SERM), or a pharmaceutically acceptable salt thereof to said subject.

In another embodiment, this invention provides a method of preventing, treating, suppressing, inhibiting or reducing the risk of developing skeletal-related events (SRE) in a human male subject suffering from prostate cancer, said method comprising the step of administering 80 mg per day of a selective estrogen receptor modulator (SERM), or a pharmaceutically acceptable salt thereof to said subject.

In one embodiment, this invention provides a method of preventing, treating, suppressing, inhibiting or reducing the risk of developing skeletal-related events (SRE) in a subject suffering from cancer, said method comprising the step of administering toremifene, or a pharmaceutically acceptable salt thereof to said subject.

In another embodiment, this invention provides a method of preventing, treating, suppressing, inhibiting or reducing the risk of developing skeletal-related events (SRE) in a male subject suffering from prostate cancer, said method comprising the step of administering toremifene, or a pharmaceutically acceptable salt thereof to said subject.

In another embodiment, this invention provides a method of preventing, treating, suppressing, inhibiting or reducing the risk of developing skeletal-related events (SRE) in a human male subject suffering from prostate cancer, said method comprising the step of administering toremifene, or a pharmaceutically acceptable salt thereof to said subject.

In another embodiment, this invention provides a method of preventing, treating, suppressing, inhibiting or reducing the risk of developing skeletal-related events (SRE) in a human male subject suffering from prostate cancer, said method comprising the step of administering 80 mg per day of toremifene, or a pharmaceutically acceptable salt thereof to said subject.

In one embodiment, the skeletal-related events are a product of cancer therapy. In one embodiment, the skeletal-related events are a product of androgen deprivation therapy. In another embodiment, the skeletal-related events are a product of cancer metastasis. In another embodiment, the invention comprises treating any skeletal-related event with a SERM, such as toremifene, or a metabolite thereof, or compositions comprising the same. In some embodiments, the SRE is bone metastasis, or pain as a result of the same, in a subject.

In one embodiment, a SRE may comprise a pathological fracture. “Pathological fracture” refers in one embodiment to a spontaneous fracture type II. A pathological fracture arises spontaneously, without adequate trauma to account for it. The bone may have been previously damaged, by a local bone lesion (e.g., metastasis, radio-osteonecrosis, or bone tumor). In another embodiment, the methods provided herein, using the compositions provided herein are used in the treating, reducing the incidence or severity of, or reducing the risk of developing a pathological fracture as a consequence of bone lesions resulting from metastasis or bone tumor arising in one embodiment from cancer, or in another embodiment from prostate cancer. In another embodiment, the term “pathological fracture” refers to a chronic fracture, fatigue fracture, stress fracture, or other similar fractures resulting from imbalance in bone resorption-formation processes as a consequence of neoplastic processes or treatment of neoplastic processes.

According to this aspect of the invention and in one embodiment, provided herein is a method of preventing, treating, suppressing, inhibiting or reducing the risk of developing fractures resulting from imbalance in bone resorption-formation processes as a consequence of neoplastic processes or treatment of neoplastic processes in a subject presenting neoplasia, said method comprising the step of administering to the subject a therapeutically effective amount of a composition comprising toremifene, SERM or their combination or a pharmaceutically acceptable salt thereof to said subject.

Bone pain is one of the most common complications of metastatic bone disease, resulting in one embodiment from structural damage, or periosteal irritation and nerve entrapment in other embodiments. In another embodiment, pain caused by bone metastasis may also be related to the rate of bone resorption. In one embodiment, the methods provided herein are effective in the treatment of bone pain, or in another embodiment, in the treatment of SRE as a function of bone pain as described herein.

Hypercalcemia has been recognized as a complication of malignancy and occurs in patients harbouring a variety of cancers including prostate cancer. This is of particular significance in prostate and breast cancer which are often associated with skeletal metastasis where osteolytic effects of PTHrP results in increased bone resorption and hypercalcemia. Hypercalcemia occurs in one embodiment, in patients with breast carcinoma, multiple myeloma, and squamous carcinomas of the lung and other primary sites. Accordingly, provided herein are methods and compositions for preventing, treating, suppressing, inhibiting or reducing the risk of developing hyperclacemia resulting from imbalance in bone resorption-formation processes as a consequence of neoplastic processes or treatment of neoplastic processes in a subject presenting neoplasia, said method comprising the step of administering to the subject a therapeutically effective amount of a composition comprising toremifene, SERM or their combination or a pharmaceutically acceptable salt thereof to said subject.

Osteoporosis is a systemic skeletal disease, characterized by low bone mass and deterioration of bone tissue, with a consequent increase in bone fragility and susceptibility to fracture. In osteoporotic patients, bone strength is abnormal, with a resulting increase in the risk of fracture. Osteoporosis depletes both the calcium and the protein collagen normally found in the bone, resulting in either abnormal bone quality or decreased bone density. Bones that are affected by osteoporosis can fracture with only a minor fall or injury that normally would not cause a bone fracture. The fracture can be either in the form of cracking (as in a hip fracture) or collapsing (as in a compression fracture of the spine). The spine, hips, and wrists are common areas of osteoporosis bone fractures, although fractures can also occur in other skeletal areas.

BMD is a measured calculation of the true mass of bone. The absolute amount of bone as measured by bone mineral density (BMD) generally correlates with bone strength and its ability to bear weight. By measuring BMD, it is possible to predict fracture risk in the same manner that measuring blood pressure can help predict the risk of stroke.

BMD, in one embodiment, can be measured by known bone-mineral content mapping techniques. Bone density of the hip, spine, wrist, or calcaneus may be measured by a variety of techniques. The preferred method of BMD measurement is dual-energy x-ray densitometry (DXA). BMD of the hip, antero-posterior (AP) spine, lateral spine, and wrist can be measured using this technology. Measurement at any site predicts overall risk of fracture, but information from a specific site is the best predictor of fracture at that site. Quantitative computerized tomography (QCT) is also used to measure BMD of the spine. See for example, “Nuclear Medicine: “Quantitative Procedures”. by Wahner H W, Dunn W L, Thorsen H C, et al, published by Toronto Little, Brown & Co., 1983, (see pages 107-132). An article entitled “Assessment of Bone Mineral Part 1” appeared in the Journal of Nuclear Medicine, pp 1134-1141, (1984). Another article entitled “Bone Mineral Density of The Radius” appeared in Vol.26, No. 11, (1985) Nov. Journal of Nuclear Medicine at pp 13-39. Abstracts on the use of gamma cameras for bone-mineral content measurements are (a) S. Hoory et al, Radiology, Vol. 157(P), p. 87 (1985), and (b) C. R. Wilson et al, Radiology, Vol. 157(P), p. 88 (1985).

The present invention provides a safe and effective method for treating, preventing, suppressing, inhibiting or reducing the risk of developing SREs, in particular, androgen-deprivation induced SREs and is useful for treating SRE particularly useful for treating male subjects suffering from prostate cancer having an elevated risk of developing androgen-deprivation induced SREs.

SERMs, and in particular, toremifene, at the doses described herein is effective at treating, suppressing or inhibiting osteopenia accompanied by bone loss. “Osteopenia” refers to decreased calcification or density of bone. This is a term which encompasses all skeletal systems in which such a condition is noted.

The invention includes the administration of “pharmaceutically acceptable salts” of SERMs and toremifene. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

Pharmaceutical Compositions

In one embodiment, the methods of the present invention comprise administering a pharmaceutical composition comprising a SERM, which in one embodiment is toremifene at a dosage which results in the delivery of 80 mg to the subject, in single dose units. In one embodiment, the pharmaceutical composition is administered to a male human subject suffering from prostate cancer; for treating and/or preventing androgen-deprivation induced SREs; for suppressing or inhibiting androgen-deprivation induced SREs; and/or for reducing the risk of developing androgen-deprivation induced SREs in a male subject.

As used herein, “pharmaceutical composition” means a “therapeutically effective amount” of the active ingredient, i.e. a SERM such as toremifene, together with a pharmaceutically acceptable carrier or diluent. A “therapeutically effective amount” as used herein refers to that amount which provides a therapeutic effect for a given condition and administration regimen.

The pharmaceutical compositions containing a SERM, such as toremifene can be administered to a subject by any method known to a person skilled in the art, such as parenterally, paracancerally, transmucosally, transdermally, intramuscularly, intravenously, intradermally, subcutaneously, intraperitonealy, intraventricularly, intracranially, intravaginally or intratumorally.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In one embodiment of the present invention, a SERM such as toremifene is formulated in a capsule. In accordance with this embodiment, the compositions of the present invention comprise, in addition to a SERM such as toremifene and the inert carrier or diluent, a hard gelating capsule.

Further, in another embodiment, the pharmaceutical compositions are administered by intravenous, intraarterial, or intramuscular injection of a liquid preparation. Suitable liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intraarterially, and are thus formulated in a form suitable for intraarterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, creams, lotions, drops and the like. For topical administration, a SERM such as toremifene is formulated in a composition comprising a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of a SERM such as toremifene over a period of time.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp.353-365 (1989); Lopez-Berestein, ibid., pp. 317-327; see generally ibid).

As used herein “pharmaceutically acceptable carriers or diluents” are well ( known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents(e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants.

In one embodiment, the pharmaceutical compositions provided herein are controlled-release compositions, i.e. compositions in which a SERM such as toremifene is released over a period of time after administration. Controlled- or sustained-release compositions include formulation in lipophilic depots (e.g. fatty acids, waxes, oils). In another embodiment, the composition is an immediate-release composition, i.e. a composition in which a SERM such as toremifene is released immediately after administration.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled-release systems are discussed in the review by Langer (Science 249:1527-1533 (1990).

The compositions may also include incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention is the modification of a SERM such as toremifene by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, a SERM such as toremifene is mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, a SERM such as toremifene is converted into a solution, suspension, or emulsion, if desired with the substances customary and suitable for this purpose, for example, solubilizers or other.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of a SERM such as toremifene are pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic: acid, oxalic acid, citric acid, tartaric acid, carbonic acid or phosphoric acid.

As used herein, the term “treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity, advancing, growing or becoming worse. As used herein, the term “recurrence” means the return of a disease after a remission.

In one embodiment, “treating” refers to both therapeutic treatment and prophylactic or preventative measures, wherein the object is to prevent or lessen SREs as described hereinabove. Thus, in one embodiment, treating may include directly affecting or curing, suppressing, inhibiting, preventing, reducing the severity of, delaying the onset of, reducing symptoms associated with the SREs, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

As used herein, the term “administering” refers to bringing a subject in contact with an anti-estrogen compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

As defined herein, “contacting” means that a SERM such as toremifene is introduced into a sample containing the enzyme in a test tube, flask, tissue culture, chip, array, plate, microplate, capillary, or the like, and incubated at a temperature and time sufficient to permit binding of the SERM to the enzyme. Methods for contacting the samples with a SERM such as toremifene or other specific binding components are known to those skilled in the art and may be selected depending on the type of assay protocol to be run. Incubation methods are also standard and are known to those skilled in the art.

In another embodiment, the term “contacting” means that a SERM such as toremifene is introduced into a subject receiving treatment, and a SERM such as toremifene is allowed to come in contact with the estrogen receptor in vivo.

In one embodiment, the methods of the present invention comprise administering a SERM such as toremifene as the sole active ingredient. However, also encompassed within the scope of the present invention are methods for hormone therapy, for treating SRE, for delaying the progression of SRE, and for preventing and/or treating the recurrence of SRE, which comprise administering a SERM such as toremifene at a dose of about 80 mg per day, in combination with one or more therapeutic agents. These agents include, but are not limited to: LHRH analogs, reversible antiandrogens (such as bicalutamide or flutamide), additional anti-estrogens, anticancer drugs, 5-alpha reductase inhibitors, aromatase inhibitors, progestins, selective androgen receptor modulators (SARMS) or agents acting through other nuclear hormone receptors.

Thus, in one embodiment, the methods of the present invention include using compositions and pharmaceutical compositions providing a SERM such as toremifene at an effective dose and further comprising an LHRH analog. In another embodiment, the methods of the present invention include using compositions and pharmaceutical compositions providing a SERM such as toremifene at an effective dose and further comprising a reversible antiandrogen.

In another embodiment, the methods of the present invention include using compositions and pharmaceutical compositions providing a SERM such as toremifene at an effective dose and further comprising an anti-estrogen. In another embodiment, the methods of the present invention include using compositions and pharmaceutical compositions providing a SERM such as toremifene at an effective dose and further comprising with an anticancer drug. In another embodiment, the methods of the present invention include using compositions and pharmaceutical compositions providing a SERM such as toremifene at an effective dose and further comprising a 5-alpha reductase inhibitor. In another embodiment, the methods of the present invention include using compositions and pharmaceutical compositions providing a SERM such as toremifene at an effective dose and further comprising an aromatase inhibitor. In another embodiment, the methods of the present invention include using compositions and pharmaceutical compositions providing a SERM such as toremifene at an effective dose and further comprising a progestin. In another embodiment, the methods of the present invention include using compositions and pharmaceutical compositions comprising providing a SERM such as toremifene at an effective dose and further comprising a SARM. In another embodiment, the methods of the present invention include using compositions and pharmaceutical compositions providing a SERM such as toremifene at an effective dose and further comprising an agent acting through other nuclear hormone receptors. In one embodiment, such an effective dose is 80 mg/day.

Thus, it is to be understood that in some embodiments, any of the compositions for use in this invention will comprise a SERM, such as toremifene in one embodiment, in any form or embodiment as described herein. In some embodiments, any of the compositions for use in this invention will consist of a SERM, such as toremifene in one embodiment, in any form or embodiment as described herein. In some embodiments, the compositions for use in this invention will consist essentially of a SERM, such as toremifene in one embodiment, in any form or embodiment as described herein. In some embodiments, the term “comprise” refers to the inclusion of the indicated active agent, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

The following examples are presented in order to more fully illustrate the preferred embodiments of the invention. They should in no way, however, be construed as limiting the broad scope of the invention.

### EXAMPLES

### Example 1

### Effect of Toremifene on Skeletal-Related Effects

Study Design

The safety and efficacy of toremifene in preventing bone fractures and/or skeletal-related events (SRE) in 1392 men with prostate cancer receiving androgen deprivation therapy was evaluated in a 24-month study. 80 mg of toremifene or placebo per day was administered to subjects. The proportion of subjects at 24 months with at least one new vertebral morphometric fracture was determined by blinded central review of radiographs. Frequency and severity of hot flashes, levels of fasting cholesterol (HDL, LDL, triglycerides) and gynecomastia (diameter of glandular tissue of each breast in supine patient; pain and tenderness) were also evaluated.

Included in the study were men with a histologically confirmed diagnosis of prostate cancer who have been treated with ADT for at least 6 months, who are more than 70 years of age or are at least 50 years of age and have had an orchiectomy or have received LHRHα for at least 6 months prior to the beginning of the study or have received intermittent LHRHa for at least the preceding 12 months or have a bone mineral density (BMD) of at or below the threshold outlined in Table 1.

TABLE 1
BMD threshold for subjects for inclusion in the study

| | Hologic BMD (g/cm2) | Lunar BMD (g/cm2) |
|---|---|---|
| L1-L4 | 0.926 | 1.050 |
| Femoral Neck | 0.717 | 0.840 |

The following skeletal related events (SRE) were evaluated: pathologic fractures, surgery to bone, radiation to bone, spinal cord compression, change in antineoplastic therapy, including changes in hormonal therapy, new bone metastases, and more than 7% bone loss.

Centrally read radiographs for vertebral morphometric fractures and locally read radiographs for non-vertebral, non-traumatic fractures taken at baseline, 12 and 24 months were used to evaluate pathologic fractures.

Data regarding surgery to bone was captured initially as serious adverse events/adverse events, and a complete follow-up was performed.

Data regarding radiation to bone was captured as progress notes in files.

Data regarding spinal cord compression was captured as serious adverse event/adverse event, radiograph of spine, progress notes, etc.

Data regarding changes in antineoplastic therapy was captured in con-med files.

Bone loss was evaluated using bone scans taken at baseline and 24 months and read centrally and dual energy X-ray absorptiometry (DEXA) taken at baseline, 12 and 24 months and read centrally.

For primary data analysis, the time to first SRE was evaluated in a cohort of all 1392 randomized subjects. For secondary data analysis, the proportion of subjects with at least 1 SRE was considered.

Baseline Data

Table 2 shows the age (years) of the subjects in the study:

| | |
|---|---|
| All Subjects | 76.1 ± 7.16 |
| US only | 76.3 ± 7.12 |
| Mexico only | 75.3 ± 7.78 |

Table 3 presents the number of years the subjects in the study have been on ADT:

| | |
|---|---|
| All Subjects | 3.17 ± 3.03 |
| US only | 3.35 ± 3.32 |
| Mexico only | 2.81 ± 2.31 |

Table 4 presents the percentage of subjects in the study with PSA<2.5:

| | |
|---|---|
| All Subjects | 10.0% |
| US only | 9.5% |
| Mexico only | 11.8% |

Table 5 shows the baseline vertebral morphometric fractures of the subjects in the study:

| | n | # with fracture | % with fracture |
|---|---|---|---|
| All Subjects | 1304 | 244 | 18.7% |
| US only | 1043 | 183 | 17.5% |
| Mexico only | 261 | 61 | 23.4% |

Table 6 shows the baseline bone metastases (hot spots located in dual energy X-ray absorptiometry (DEXA) scan areas hip and spine) of the subjects in the study:

| | n | # with hot spot | % with hot spot |
|---|---|---|---|
| All Subjects | 1130 | 191 | 16.9% |
| US only | 909 | 135 | 14.9% |
| Mexico only | 221 | 56 | 25.3% |

Table 7 shows baseline percentage of osteopenic subjects:

| | % with bone loss |
|---|---|
| All Subjects | 75.0% |
| US only | 74.0% |
| Mexico only | 79.9% |

Table 8 shows baseline percentage of osteoporotic subjects:

| | % osteoporotic |
|---|---|
| All Subjects | 17.6% |
| US only | 17.3% |
| Mexico only | 18.7% |

Results

Subjects treated with Toremifene were evaluated for a series of bone-related events, outlined in Table 9.

TABLE 9

| Event | Observed | Total Expected |
|---|---|---|
| Pathologic fractures | 66 | 123 |
| Surgery to bone | 1 | 2 |
| Radiation to bone | 1? | 2 |
| Spinal cord compression | 1? | 2 |
| Change in antineoplastic therapy | 42 | 64 |
| >7% bone loss | 44 | 81 |
| Mew Bone Metastasis | ? | 160 |
| TOTAL EVENTS | 155 | 434 |

Of the 66 pathologic fractures, 24 were new and worsening vertebral morphometric fractures, while 42 were non-vertebral morphometric fractures (˜3 to 4% per year). The ratio of non-vertebral to vertebral fractures is ˜2:1. This is a similar ratio to other reported observations.

Aggregate Vertebral Monphometric Fracture Rate

Subjects were evaluated for their aggregate vertebral
morphometric fracture rates (Table 10).

| | n | % fractures | # fractures |
|---|---|---|---|
| 12 month evaluated | 704 | 2.4% | 17 |
| 12 month pool | 311 | 2.4% | 8 |
| 24 month evaluated | 154 | 3.9% | 6 |
| 24 month “pool” | 576 | 3.9% | 23 |
| EDC 12 to 24 month “pool” | 60 | 1.95% | 1 |
| TOTAL | 1015^{a} | 5.4% | 55 |

^{a}1015/1392 evaluable subjects with on study bone assessments (73% of subjects)

The aggregate vertebral morphometric fracture rate was 2.4% (17/704) at 12 months and 3.9% (6/154) at 24 months. There were five unconfirmed vertebral morphometric fractures, which if confirmed, would increase year one to 3.1% of subjects suffering fractures and increase the total number of fractures to 60.

The projected number non-vertebral, non-traumatic fractures in year2 is approximately 26, based on the assumption of a 3.5% event rate in 730 patients expected to complete 24 months. This will bring the total projected number of clinical fractures to approximately 68.

Change in Antineoplastic Therapy

42 subjects with changes in antineoplastic therapy have been observed due to early terminations due to “disease progression”. 22 additional subjects with changes in antineoplastic therapy are expected based on the 3% “disease progressions” that were observed (42/1392) multiplied by the 730 patients expected to complete the second year of treatment.

>7% Bone Loss

There were 23 confirmed and 8 unconfirmed patients with >7% bone loss at 12 months out of 939 patients evaluated (2.4% to 3.3%)

There were 13 confirmed patients with >7% bone loss at 24 months with >7% bone loss out of 202 patients evaluated (6.4%)

It is projected that 3 additional patients at 12 months and 34 at 24 months will be evaluated with >7% bone loss, bringing the total expected incidence of bone loss to 81, assuming that the 8 unconfirmed cases at 12 months are confirmed.

New Bone Metastases

The projected total of new bone metastases is 11% per year in the placebo group with 160 total expected events based on the following assumptions: the treatment effect is a 33% reduction in events; 34% of the patients had bone metastases at baseline; 1015 assessments will be performed in the first year and 730 in the second year; patients started ADT at time of diagnosis

In summary, only 31.2% (434/1392) of prostate cancer subjects on androgen deprivation therapy are expected to demonstrate an SRE after toremifene treatment compared to approximately 44% of prostate cancer subjects given a placebo in other studies.

Toremifene citrate produced statistically significant and clinically meaningful changes in SRE in men treated with ADT for prostate cancer. Toremifene citrate is beneficial for reducing SRE in subjects with cancer.

It will be appreciated by a person skilled in the art that the present invention is not limited by what has been particularly shown and described hereinabove. Rather, the scope of the invention is defined by the claims which follow:

What is claimed is:

1. A method of preventing, treating, suppressing, inhibiting or reducing the risk of, reducing the incidence of, ameliorating symptoms of, delaying progression of, diminishing pathogenesis of a skeletal-related event (SRE) in a subject suffering from cancer, said method comprising the step of administering toremifene, or a pharmaceutically acceptable salt thereof to said subject.

2. The method according to claim 1, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

3. The method according to claim 2, wherein said pharmaceutical composition is a pellet, a tablet, a capsule, a solution, a suspension, an emulsion, an elixir, a gel, a cream, a suppository or a parenteral formulation.

4. The method according to claim 1, wherein said subject is human.

5. The method according to claim 1, wherein said subject is male.

6. The method according to claim 1, wherein said cancer is prostate cancer.

7. The method according to claim 1, wherein said SERM is toremifene.

8. The method according to claim 7, wherein said toremifene is administered at a dose of 80 mg per day.

9. The method according to claim 1, wherein said skeletal-related event (SRE) comprises a pathologic fracture, a necessity for surgery of the bone, a necessity for radiation of the bone, spinal cord compression, new bone metastasis, bone loss, or a combination thereof.

10. The method according to claim 9, wherein said bone loss is greater than 7%.

11. The method according to claim 1, wherein said skeletal-related event is a product of cancer therapy.

12. The method according to claim 11, wherein said cancer therapy is androgen deprivation therapy.

13. The method according to claim 1, wherein said subject demonstrates enhanced cancer pathogenesis proximal to, or prior to administering said selective estrogen receptor modulator (SERM).

14. The method according to claim 13, wherein said subject requires a change in antineoplastic therapy.

15. The method according to claim 14, wherein the antineoplastic therapy is changed as a function of disease pathogenesis.

16. The method according to claim 14, wherein the antineoplastic therapy is changed as in response to pain in the subject.

17. A method of preventing, treating, suppressing, inhibiting or reducing the risk of, reducing the incidence of, ameliorating symptoms, delaying progression, diminishing pathogenesis of developing a skeletal-related event (SRE) in a male subject suffering from prostate cancer, said method comprising the step of administering a selective estrogen receptor modulator (SERM), or a pharmaceutically acceptable salt thereof to said subject.

18. The method according to claim 17, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

19. The method according to claim 18, wherein said pharmaceutical composition is a pellet, a tablet, a capsule, a solution, a suspension, an emulsion, an elixir, a gel, a cream, a suppository or a parenteral formulation.

20. The method according to claim 17, wherein said subject is human.

21. The method according to claim 17, wherein said SERM is toremifene.

22. The method according to claim 21, wherein said toremifene is administered at a dose of 80 mg per day.

23. The method according to claim 17, wherein said skeletal-related event (SRE) comprises a pathologic fracture, a necessity for surgery of the bone, a necessity for radiation of the bone, spinal cord compression, new bone metastasis, bone loss, or a combination thereof.

24. The method according to claim 23, wherein said bone loss is greater than 7%.

25. The method according to claim 17, wherein said skeletal-related event is a product of cancer therapy.

26. The method according to claim 25, wherein said cancer therapy is androgen deprivation therapy.

27. The method according to claim 17, wherein said subject demonstrates enhanced cancer pathogenesis proximal to, or prior to administering said selective estrogen receptor modulator (SERM).

28. The method according to claim 27, wherein said subject requires a change in antineoplastic therapy.

29. The method according to claim 23, wherein the necessity for surgery, the necessity for radiation of the bone, or their combination, is done in order to treat pain in the subject as a result of said prostate cancer.

30. The method according to claim 28, wherein the antineoplastic therapy is changed as a function of disease pathogenesis.

31. The method according to claim 28, wherein the antineoplastic therapy is changed as in response to pain in the subject.

32. A method of preventing, treating, suppressing, inhibiting or reducing the risk of, reducing the incidence of, ameliorating symptoms, delaying progression, diminishing pathogenesis of developing a skeletal-related event (SRE) in a male subject suffering from prostate cancer, said method comprising the step of administering toremifene, or a pharmaceutically acceptable salt thereof to said subject.

33. The method according to claim 32, wherein said administering comprises intravenously, intraarterially, or intramuscularly injecting to said subject said pharmaceutical composition in liquid form; subcutaneously implanting in said subject a pellet containing said pharmaceutical composition; orally administering to said subject said pharmaceutical composition in a liquid or solid form; or topically applying to the skin surface of said subject said pharmaceutical composition.

34. The method according to claim 33, wherein said pharmaceutical composition is a pellet, a tablet, a capsule, a solution, a suspension, an emulsion, an elixir, a gel, a cream, a suppository or a parenteral formulation.

35. The method according to claim 32, wherein said subject is human.

36. The method according to claim 32, wherein said toremifene is administered at a dose of 80 mg per day.

37. The method according to claim 32, wherein said skeletal-related event (SRE) comprises a pathologic fracture, a necessity for surgery of the bone, a necessity for radiation of the bone, spinal cord compression, new bone metastasis, bone loss, or a combination thereof.

38. The method according to claim 37, wherein said bone loss is greater than 7%.

39. The method according to claim 32, wherein said skeletal-related events are a product of cancer therapy.

40. The method according to claim 39, wherein said cancer therapy is androgen deprivation therapy.

41. The method according to claim 32, wherein said subject demonstrates enhanced cancer pathogenesis proximal to, or prior to administering toremifene.

42. The method according to claim 41, wherein said subject requires a change in antineoplastic therapy.

43. The method according to claim 42, wherein the antineoplastic therapy is changed as a function of disease pathogenesis.

44. The method according to claim 42, wherein the antineoplastic therapy is changed as in response to pain in the subject.
